SEQUENCE LISTING (5 sequences)

SEQ ID NO: 1 - protein, 396 aa, Mus musculus
  MISC_FEATURE (1)..(19): VH5 leader sequence
MGSTAILGLLLAVLQGGRACKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGGSQEANALPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 2 - protein, 377 aa, Mus musculus
  MISC_FEATURE (222)..(226): Linker sequence
CKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGGSQEANALPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 3 - DNA, 1188 bp, Mus musculus
atggggtcaaccgccatccttggcctcctcctggctgtcctgcagggagggcgcgcctgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaaggaggcggtggaagccaggaggcaaatgcgctgcccgtcaacacccggtgcaagcttgaggtgtccaacttccagcagccgtacatcgtcaaccgcacctttatgctggccaaggaggccagccttgcagataacaacacagacgtccggctcatcggggagaaactgttccgaggagtcagtgctaaagatcagtgctacctgatgaagcaggtgctcaacttcaccctggaagacgttctgctcccccagtcagacaggttccagccctacatgcaggaggtggtacctttcctgaccaaactcagcaatcagctcagctcctgtcacatcagcggtgacgaccagaacatccagaagaatgtcagaaggctgaaggagacagtgaaaaagcttggagagagtggagagatcaaggcgattggggaactggacctgctgtttatgtctctgagaaatgcttgcgtc

SEQ ID NO: 4 - protein, 383 aa, Homo sapiens
  MISC_FEATURE (152)..(156): Linker sequence
QGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 383 aa, Homo sapiens
  MISC_FEATURE (228)..(232): Linker sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

(19) United States
(12) Patent Application Publication
(10) Pub. No.: US 2013/0121959 A1
(43) Pub. Date: May 16, 2013

(54) IL-22-FC AND HEPCIDIN ACTIVITY

(71) Applicants: Joseph R. Maxwell, Seattle, WA (US); Jamesb B. Rottman, Sudbury, MA (US); Carole L. Smith, Seattle, WA (US); Keegan Cooke, Ventura, CA (US); Tara Arvedson, Simi Valley, CA (US); Barbara J. Sasu, Westlake Village, CA (US)

(72) Inventors: Joseph R. Maxwell, Seattle, WA (US); Jamesb B. Rottman, Sudbury, MA (US); Carole L. Smith, Seattle, WA (US); Keegan Cooke, Ventura, CA (US); Tara Arvedson, Simi Valley, CA (US); Barbara J. Sasu, Westlake Village, CA (US)

(21) Appl. No.: 13/522,001

(22) PCT Filed: Jan. 10, 2011
(86) PCT No.: PCT/US11/20678

§ 371 Date: Dec. 3, 2012

Related U.S. Application Data
(60) Provisional application No. 61/294,595, filed on Jan. 13, 2010.

(51) Int. Cl. A61K 39/395

(52) U.S. Cl. CPC A61K 39/395
USPC 424/85.2; 530/387.3; 530/387.9

(57) ABSTRACT

The invention relates to an IL-22-Fc molecule to regulate hepcidin activity/expression and/or iron export from a cell.

### CROSS REFERENCE TO RELATED APPLICATIONS

This application claims the benefit of U.S. Provisional Application No. 61/294,595, filed Jan. 13, 2010, which is hereby incorporated by reference.

### FIELD OF THE INVENTION

The invention relates to an IL-22-Fc molecule to regulate hepcidin activity/expression and/or iron export from a cell.

### BACKGROUND OF THE INVENTION

Iron is an essential trace element required for growth and development of all living organisms. Iron content in mammals is regulated by controlling iron absorption, iron recycling, and release of iron from the cells in which it is stored. Iron is absorbed predominantly in the duodenum and upper jejunum by enterocytes. A feedback mechanism exists that enhances iron absorption in individuals who are iron deficient, and that reduces iron absorption in individuals with iron overload (Andrews, Ann. Rev. Genomics Hum. Genet., 1:75, 2000; Philpott, Hepatology, 35:993, 2002; Beutler et al., Drug-Metab. Dispos., 29:495, 2001). Iron is recycled from degraded red cells by reticuloendothelial macrophages in bone marrow, hepatic Kupffer cells and the spleen. Iron release is controlled by ferroportin, a major iron export protein located on the cell surface of enterocytes, macrophages and hepatocytes, the main cells capable of releasing iron into plasma. Hepcidin binds to ferroportin and decreases ferroportin's functional activity by causing it to be internalized from the cell surface and degraded. (Nemeth et al., Science, 306:2090-3, 2004; De Domenico et al., Mol. Biol. Cell., 8:2569-2578, 2007).

Hepcidin is the key signal regulating iron homeostasis (Philpott, Hepatology 35:993, 2002; Nicolas et al., Proc. Natl. Acad. Sci. USA, 99:4396, 2002). High levels of human hepcidin result in reduced iron levels, and vice versa. Mutations in the hepcidin gene which result in lack of hepcidin activity are associated with juvenile hemochromatosis, a severe iron overload disease (Roetto et al., Nat. Genet., 33:21-22, 2003). Studies in mice have also demonstrated a role of hepcidin in control of normal iron homeostasis (Nicolas et al., Nat. Genet., 34:97-101, 2003; Nicolas et al., Proc. Natl. Acad. Sci. USA, 99:4596-4601, 2002; Nicolas et al., Proc. Natl. Acad. Sci. USA, 98:8780-8785, 2001).

Interleukin-22 (IL-22) is a class II cytokine that is up-regulated in T cells. One function of IL-22 is to enhance the innate immunity of peripheral tissues by inducing the expression of anti-microbial peptides (Wolk et al., Immunity, 21:241-54, 2004; Boniface et al., J. Immunol., 174:3695-3702, 2005). Other studies have shown that expression of IL-22 mRNA is induced in vivo in response to LPS administration, and that IL-22 modulates parameters indicative of an acute phase response (Dumoutier L. et al., Genes Immunol., 1(8):488-494, (2000); Pittman et al., Genes and Immunity, 2:172, 2001). Taken together, these observations indicate that IL-22 plays a role in inflammation (Kotenko S. V., Cytokine & Growth Factor Reviews, 13(3):223-40, 2002). Several T cell disorders are associated with increased levels of IL-22 (Wolk et al., Immunity, 21:241-54, 2004; Ikeuchi H. et al., Arthritis Rheum., 52:1037-1046, 2005); Andoh, A. et al., Gastroenterology, 129:969-984, 2005).

An Fc fusion protein containing IL-22 has previously been prepared by fusion of the entire open reading frame of IL-22 with the Fc region of human IgG₁. (Xie et al., J. Biol. Chem., 275(40):31335-31339, 2000). This has been used to study IL-22 binding.

Prophylactic IL-22R stimulation can protect animals from dextran sulphate sodium (DSS)-induced colitis. Metabolic effects such as iron deficiency and weight loss can complicate the benefit received (Smith et al., J. Immunol., 182:38.7, 2009). In this model IL-22-Fc exacerbated weight loss and induced anemia. A better understanding of the relationship between IL-22, hepcidin and iron storage would be beneficial. This relationship is discussed in this patent.

### SUMMARY OF THE INVENTION

In various embodiments, a method of increasing hepcidin expression and/or activity in a mammal comprising stimulating the IL-22 pathway is provided.

In other embodiments, an isolated amino acid sequence comprising SEQ ID NO:1, SEQ ID NO:2, SEQ ID NO:4 or SEQ ID NO:5 is provided.

In yet other embodiments, an isolated amino acid sequence at least 95% identical to SEQ ID NO:1, SEQ ID NO:2, SEQ ID NO:4 or SEQ ID NO:5 is provided.

In other embodiments an agonist of an IL-22 receptor is provided, wherein said agonist increases hepcidin expression or hepcidin activity.

### BRIEF DESCRIPTION OF THE DRAWINGS

FIG. 1: Serum iron is decreased in mice treated with IL-22-Fc.

FIG. 2: Serum hepcidin levels are elevated in mice treated with IL-22-Fc.

FIG. 3. Red blood cell and reticulocyte parameters in mice treated with IL-22-Fc.

FIG. 4: Iron accumulation in macrophage-rich regions of the spleen.

FIG. 5: Weight changes in mice treated with IL-22-Fc

FIG. 6: Changes in erythrocyte parameter following treatment with IL-22-Fc.

FIG. 7: IL-22-Fc anemia induced independently of IL-6

FIG. 8: IL-22-Fc reduces blood hemoglobin and mean corpuscular hemoglobin which is reversed by hepcidin blockage.

FIG. 9: IL-22-Fc reduces serum iron and increases iron stored in the spleen which is reversed by hepcidin blockage.

### DETAILED DESCRIPTION

In various embodiments, a method of increasing hepcidin expression and/or activity in a mammal comprising stimulating the IL-22 pathway is provided. The mammal can be a human. The stimulating can comprise treating with IL-22-Fc. In some embodiments, the IL-22-Fc can comprise an amino acid sequence from the group of SEQ ID NO:1, SEQ ID NO:2, SEQ ID NO:4 or SEQ ID NO:5. In yet other embodiments, the isolated amino acid sequence can be at least 95% identical to SEQ ID NO:1, SEQ ID NO:2, SEQ ID NO:4 or SEQ ID NO:5.

In various embodiments, the method of increasing comprises using an Fc fused to either the N- or C-terminus of IL-22. In other embodiments the increasing of hepcidin expression or activity can result in limiting iron uptake in a mammal. The iron uptake can be limited in hereditary or non-hereditary hemochromatosis, thalassemia, hemolytic anemias and other iron-loading hematological disorders such as myelodysplastic syndrome.

In various embodiments, an isolated monoclonal antibody that binds the amino acid sequence of SEQ ID NO:1, SEQ ID NO:2, SEQ ID NO:4 or SEQ ID NO:5 is provided. In yet other embodiments, the isolated amino acid sequence can be at least 95% identical to SEQ ID NO:1, SEQ ID NO:2, SEQ ID NO:4 or SEQ ID NO:5.

In various embodiments an agonist of an IL-22 receptor, wherein said agonist increases hepcidin expression or hepcidin activity. The agonist can comprise IL-22-Fc. The Fc can be fused at the N- or C-terminal In some embodiments, the IL-22-Fc can comprise an amino acid sequence from the group of SEQ ID NO:1, SEQ ID NO:2, SEQ ID NO:4 or SEQ ID NO:5. In yet other embodiments, the isolated amino acid sequence can be at least 95% identical to SEQ ID NO:1, SEQ ID NO:2, SEQ ID NO:4 or SEQ ID NO:5.

The foregoing summary is not intended to define every aspect or embodiment of the invention, and additional aspects may be described in other sections. The entire document is intended to be related as a unified disclosure, and it should be understood that all combinations of features described herein may be contemplated, even if the combination of features is not found together in the same sentence, or paragraph, or section of this document.

In addition to the foregoing, as an additional aspect, all embodiments narrower in scope in any way than the variations defined by specific paragraphs herein can be included in this patent. For example, certain aspects are described as a genus, and it should be understood that every member of a genus can be, individually, an embodiment. Also, aspects described as a genus or selecting a member of a genus should be understood to embrace combinations of two or more members of the genus. It should be understood that while various embodiments in the specification are presented using “comprising” language, under various circumstances, a related embodiment may also be described using “consisting of” or “consisting essentially of” language.

It is to be noted that the term “a” or “an”, refers to one or more, for example, “an immunoglobulin molecule,” is understood to represent one or more immunoglobulin molecules. As such, the terms “a” (or “an”), “one or more,” and “at least one” can be used interchangeably herein.

It should also be understood that when describing a range of values, the characteristic being described could be an individual value found within the range. For example, “a pH from about pH 4 to about pH 6,” could be, but is not limited to, pH 4, 4.2, 4.6, 5.1 5.5 etc. and any value in between such values. Additionally, “a pH from about pH 4 to about pH 6,” should not be construed to mean that the pH in question varies 2 pH units from pH 4 to pH 6, but rather a value may be picked from within a two pH range for the pH of the solution.

In some embodiments, when the term “about” is used, it means the recited number plus or minus 5%, 10%, 15% or more of that recited number. The actual variation intended is determinable from the context.

The following definitions are intended to assist in understanding the various embodiments.

The terms “peptide,” “polypeptide” and “protein” each refer to a molecule comprising two or more amino acid residues joined to each other by peptide bonds. These terms encompass, e.g., native and artificial proteins, protein fragments and polypeptide analogs (such as muteins, variants, and fusion proteins) of a protein sequence as well as post-translationally, or otherwise covalently or non-covalently, modified proteins. A peptide, polypeptide, or protein may be monomeric or polymeric. The terms are used interchangeably, however, the term peptide can sometimes be used to describe a short amino acid sequence.

“Recombinant proteins” refer to proteins produced by recombinant DNA techniques, i.e., produced from cells, microbial or mammalian, transformed by an exogenous DNA construct encoding the desired protein. Proteins expressed in most bacterial cultures will be free of glycan. Proteins expressed in yeast may have a glycosylation pattern different from that expressed in mammalian cells.

A “fusion protein” should be understood to be a protein created by joining parts of at least two different proteins. A fusion protein can be made by joining two different genes that originally coded separate proteins, e.g., IL-22 and an Fc, thus forming an IL-22-Fc fusion protein. The IL-22-Fc fusion protein can have the Fc portion of the protein attached directly or via a linker to the IL-22 portion of the protein. The Fc portion of the fusion protein can be attached at either the amino or carboxyl end of the IL-22-Fc fusion protein. Fusion proteins can be made from mammalian proteins, for example, mouse or human proteins.

A DNA “coding sequence” is a DNA sequence which is transcribed into mRNA and translated into a polypeptide in a host cell when placed under the control of appropriate regulatory sequences. The boundaries of the coding sequence are determined by a start codon at the 5′ N-terminus and a translation stop codon at the 3′ C-terminus. A coding sequence can include prokaryotic sequences, cDNA from eukaryotic mRNA, genomic DNA sequences from eukaryotic DNA, and synthetic DNA sequences. A transcription termination sequence will usually be located 3′ to the coding sequence.

“Nucleotide sequence” is a heteropolymer of deoxyribonucleotides (bases adenine, guanine, thymine, or cytosine). DNA sequences encoding proteins can be assembled from synthetic cDNA-derived DNA fragments or isolated naturally-found DNA and short oligonucleotide linkers to provide a synthetic gene that is capable of being expressed in a recombinant expression vector. In discussing the structure of particular double-stranded DNA molecules, sequences may be described according to the normal convention of giving only the sequence in the 5′ to 3′ direction along the nontranscribed strand of cDNA.

“Recombinant expression vector” is a replicable DNA construct used either to amplify or to express DNA encoding a protein of interest, e.g., an IL-22-Fc fusion protein. An expression vector can contain DNA control sequences and a coding sequence. DNA control sequences include promoter sequences, ribosome binding sites, polyadenylation signals, transcription termination sequences, upstream regulatory domains and enhancers. Recombinant expression systems can express IL-22-Fc upon induction of regulatory elements.

“Substantially similar functional activity” and “substantially the same biological function or activity” means that the degree of biological activity that is within about 30% to 100% or more of that biological activity demonstrated by the polypeptide to which it is being compared when the biological activity of each polypeptide is determined by the same procedure or assay.

In various embodiments, “variants” are provided. Included within variants are insertional, deletional, and substitutional variants. It is understood that in various embodiments a particular molecule may contain one, two or all three types of variants. Insertional and substitutional variants may contain natural amino acids, unconventional amino acids, amino acid analogs or combinations of the natural, unconventional and analogs.

In one example, insertional variants are provided wherein one or more amino acid residues, either naturally occurring or unconventional amino acids, supplement a peptide amino acid sequence. Insertions may be located at either or both termini of the protein, or may be positioned within internal regions of the amino acid sequence. Insertional variants with additional residues at either or both termini can include, for example, fusion proteins and proteins including amino acid tags or labels. Insertional variants include polypeptides wherein one or more amino acid residues are added to the amino acid sequence, or fragment thereof.

In various embodiments, the polypeptide can include mature polypeptides wherein leader or signal sequences are removed, and the resulting proteins having additional amino terminal residues, which amino acids may be natural or non-natural. Molecules with an additional methionyl residue at amino acid position-1 (Met¹) are contemplated, as are specific binding agents with additional methionine and lysine residues at positions-2 and -1 (Met⁻²⁻Lys⁻¹⁻). Variants having additional Met, Met-Lys, Lys residues (or one or more basic residues, in general) can be useful for enhanced recombinant protein production in bacterial host cells.

In various embodiments, variants having additional amino acid residues that arise from use of specific expression systems are contemplated. For example, use of commercially available vectors that express a desired polypeptide as part of glutathione-S-transferase (GST) fusion product provides the desired polypeptide having an additional glycine residue at amino acid position-1 after cleavage of the GST component from the desired polypeptide. Variants which result from expression in other vector systems are also contemplated, including those wherein poly-histidine tags are incorporated into the amino acid sequence, generally at the carboxy and/or amino terminus of the sequence.

Insertional variants also include fusion proteins wherein the amino and/or carboxy termini is fused to another polypeptide, a fragment thereof or amino acids which are not generally recognized to be part of any specific protein sequence. Examples of such fusion proteins are immunogenic polypeptides or proteins with long circulating half lives, such as immunoglobulin constant regions.

This type of insertional variant may have all or a substantial portion of the native molecule, linked at the N- or C-terminus, to all or a portion of a second polypeptide. For example, fusion proteins may employ leader sequences from other species to permit the recombinant expression of a protein in a heterologous host. Another useful fusion protein includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

There are various commercially available fusion protein expression systems that may be used to make fusion proteins. Particularly useful systems include but are not limited to the glutathione-S-transferase (GST) system (Pharmacia), the maltose binding protein system (NEB, Beverley, Mass.), the FLAG system (IBI, New Haven, Conn.), and the 6×His system (Qiagen, Chatsworth, Calif.). These systems are capable of producing recombinant peptides bearing only a small number of additional amino acids, which are unlikely to significantly affect the activity of a polypeptide of interest. For example, both the FLAG system and the 6×His system add only short sequences, both of which are known to be poorly antigenic and which do not adversely affect folding of a polypeptide to its native conformation. Another N-terminal possible fusion is the fusion of a Met-Lys dipeptide at the N-terminal region of the protein or peptides. Such a fusion may produce beneficial increases in protein expression or activity.

In various embodiments, deletion variants are provided wherein one or more amino acid residues in a peptide are removed. Deletions can be effected at one or both termini, or from removal of one or more residues within the amino acid sequence. Deletion variants can include all fragments of a peptide.

In still another aspect, substitution variants of peptides are provided. Substitution variants include those peptides wherein one or more amino acid residues are removed and replaced with one or more alternative amino acids, which may be naturally occurring or non-naturally occurring. Substitutional variants generate peptides that are “similar” to the original peptide, in that the two molecules have a certain percentage of amino acids that are identical. Substitution variants include substitutions of 1, 2, 3, 4, 5, 6, 7, 8, 9, 10, 15, 20, 25 or 30 amino acids within a peptide, wherein the number of substitutions may be up to ten percent or more, of the amino acids of the peptide. In one aspect, the substitutions are conservative in nature, however, various embodiments embrace substitutions that are also non-conservative and can include unconventional amino acids.

Identity and similarity of related peptides can be readily calculated by known methods. Such methods include, but are not limited to, those described in Computational Molecular Biology, Lesk, A. M., ed., Oxford University Press, New York (1988); Biocomputing: Informatics and Genome Projects, Smith, D. W., ed., Academic Press, New York (1993); Computer Analysis of Sequence Data, Part 1 Griffin, A. M., and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., M. Stockton Press, New York (1991); and Carillo et al., SIAM J. Applied Math. 48:1073 (1988).

In various embodiments, a polypeptide can have a percent identity to another polypeptide. The percent identity of two polypeptides can be about 100%, 99%, 98%, 97%, 96%, 95%, 94%, 93%, 92%, 91% 90%, 89%, 88%, 87%, 86%, 85%, 84%, 83%, 82%, 81%, 80%, 75%, 70%, 65%, 60% or less. For example, in an embodiment, a claimed polypeptide can be 95% identical to an IL-22-Fc polypeptide and have similar activity to the IL-22-Fc polypeptide.

Methods to determine the relatedness or percent identity of two peptides or polypeptides, or a polypeptide and a peptide, are designed to give the largest match between the sequences tested. Methods to determine identity are described in publicly available computer programs. Preferred computer program methods to determine identity between two sequences include, but are not limited to, the GCG program package, including GAP (Devereux et al. Nucl. Acid. Res., 12:387 (1984); Genetics Computer Group, University of Wisconsin, Madison, Wis., BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al., NCB/NLM/NIH Bethesda, Md., 20894; Altschul et al., supra, (1990)). The well-known Smith Waterman algorithm may also be used to determine identity.

Certain alignment schemes for aligning two amino acid sequences may result in the matching of only a short region of the two sequences and this small aligned region may have very high sequence identity even though there is no significant relationship between the two full-length sequences. For example, in certain embodiments, the selected alignment method (GAP program) will result in an alignment that spans at least ten percent of the full length of the target polypeptide being compared, i.e. at least 40 contiguous amino acids where sequences of at least 400 amino acids are being compared, 30 contiguous amino acids where sequences of at least 300 to about 400 amino acids are being compared, at least 20 contiguous amino acids where sequences of 200 to about 300 amino acids are being compared, and at least 10 contiguous amino acids where sequences of about 100 to 200 amino acids are being compared.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). In certain embodiments, a gap opening penalty (which is typically calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see Dayhoff et al., Atlas of Protein Sequence and Structure, 5(3)(1978) for the PAM 250 comparison matrix; Henikoff et al., Proc. Natl. Acad. Sci. USA, 89:10915-10919 (1992) for the BLOSUM 62 comparison matrix) can also used by the algorithm.

In certain embodiments, the parameters for a polypeptide sequence comparison can include the following:

Algorithm: Needleman et al., J. Mol. Biol., 48:443-453 (1970);

Comparison matrix: BLOSUM 62 from Henikoff et al. supra (1992);

Gap Penalty: 12

Gap Length Penalty: 4

Threshold of Similarity: 0

The GAP program may be useful with the above parameters. In certain embodiments, the aforementioned parameters can be the default parameters for polypeptide comparisons (along with no penalty for end gaps) using the GAP algorithm.

In certain other embodiments, the parameters for polynucleotide molecule sequence (as opposed to an amino acid sequence) comparisons include the following:

Algorithm: Needleman et al., supra, (1970);

Comparison matrix: matches=+10, mismatch=0

Gap Penalty: 50

Gap Length Penalty: 3

The GAP program may also be useful with the above parameters. The aforementioned parameters can be the default parameters for polynucleotide molecule comparisons.

Other exemplary algorithms, gap opening penalties, gap extension penalties, comparison matrices, thresholds of similarity, etc. may be used, including those set forth in the Program Manual, Wisconsin Package, Version 9, September, 1997. The particular choices to be made will be apparent to those of skill in the art and will depend on the specific comparison to be made, such as DNA-to-DNA, protein-to-protein, protein-to-DNA; and additionally, whether the comparison is between given pairs of sequences (in which case GAP or BestFit are generally used) or between one sequence and a large database of sequences (in which case FASTA or BLASTA are generally used).

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)).

It will be appreciated that amino acid residues can be divided into classes based on their common side chain properties:

Neutral Hydrophobic: Alanine (Ala; A), Valine (Val; V), Leucine (Leu; L), Isoleucine (Ile; I), Proline (Pro; P), Tryptophan (Trp; W), Phenylalanine (Phe; F), and Methionine (Met, M).

Neutral Polar: Glycine (Gly; G); Serine (Ser; S), Threonine (Thr; T), Tyrosine (Tyr; Y), Cysteine (Cys; C), Glutamine (Glu; Q), Asparagine (Asn; N), and Norleucine.

Acidic: Aspartic Acid (Asp; D), Glutamic Acid (Glu; E).

Basic: Lysine (Lys; K), Arginine (Arg; R), Histidine (His; H). See Lewin, B., Genes V, Oxford University Press (1994), p. 11.

Conservative amino acid substitutions may encompass unconventional amino acid residues, which are typically incorporated by chemical peptide synthesis rather than by synthesis in biological systems. These include, without limitation, peptidomimetics and other reversed or inverted forms of amino acid moieties. Non-conservative substitutions may involve the exchange of a member of one of the classes for a member from another class.

In making changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids may be substituted for other amino acids having a similar hydropathic index or score and still retain a similar biological activity. In making changes based upon the hydropathic index, in certain embodiments, the substitution of amino acids whose hydropathic indices are within .+−0.2 is included. In certain embodiments, those which are within .+−0.1 are included, and in certain embodiments, those within .+-.0.5 are included.

“Similarity” between two polypeptides may also be determined by comparing the amino acid sequence and its conserved amino acid substitutes of one polypeptide to the sequence of a second polypeptide. Such conservative substitutions include those described above in The Atlas of Protein Sequence and Structure 5 by Dayhoff (1978) and by Argos (1989) EMBO J. 8:779-785. For example, exchange of an amino acid belonging to one of the following groups for another amino acid from the same group can represent conservative changes:

Ala, Pro, Gly, Gln, Asn, Ser, Thr:

Cys, Ser, Tyr, Thr;

Val, Leu, Met, Ala, Phe;

Lys, Arg, H is;

Phe, Tyr, Trp, H is; and

Asp, Glu.

IL-22-Fc includes, but is not limited to, a polypeptide comprising the amino acid sequence as set forth in SEQ ID NOs 1, 2, 4 or 5. Additionally, IL-22-Fc can include analogs of IL-22-Fc, with 65%, 70%, 75%, 80%, 85%, 90%, 91%, 92%, 93%, 94%, 95%, 96%, 97%, 98%, or 99% identity to SEQ. ID NOs: 1, 2, 4 or 5 and still retain activity that increases hepcidin activity or hepcidin expression.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species, (3) is expressed by a cell from a different species than normally expresses the molecule, or (4) does not occur in nature. Thus, a molecule that is chemically synthesized, or synthesized in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art, depending on the molecule of interest. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification. In general, an “isolated” molecule is either (1) identified and separated from at least one contaminant with which it is ordinarily associated in a natural source (2) or otherwise distinguished from background molecules.

More specifically, as used herein, an “isolated” nucleic acid molecule or “isolated” nucleic acid sequence is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it is found in nature.

Once isolated, the DNA may be operably linked to expression control sequences or placed into expression vectors, which are then transfected into host cells that do not otherwise produce the protein of interest, in order to direct the synthesis of the protein in the recombinant host cells. An example of a recombinant protein can be a monoclonal antibody or other fusion protein. Recombinant production of antibodies or fusion proteins is well known in the art.

“Expression control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice.

Many vectors are known in the art. Vector components may include one or more of the following: a signal sequence (that may, for example, direct secretion of a polypeptide), an origin of replication, one or more selective marker genes (that may, for example, confer antibiotic or other drug resistance, complement auxotrophic deficiencies, or supply critical nutrients not available in the media), an enhancer element, a promoter, and a transcription termination sequence, all of which are well known in the art.

“Cell, cell line, and cell culture” are often used interchangeably and all such designations herein include progeny. Transformants and transformed cells include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

Exemplary host cells include prokaryote, yeast, or higher eukaryote cells (i.e., a multicellular organism). Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas, and Streptomyces. Eukaryotic cells such as filamentous fungi or yeast are suitable cloning or expression hosts for recombinant polypeptides or antibodies. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Host cells for the expression of a glycosylated polypeptide can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of a polypeptide from such cells has become routine procedure. Examples of useful mammalian host cell lines are Chinese hamster ovary cells, including CHOK1 cells (ATCC CCL61), DXB-11, DG-44, and Chinese hamster ovary cells/−DHFR (CHO, Urlaub et al., Proc. Natl. Acad. Sci. USA, 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Virol. 36: 59 (1977)]; baby hamster kidney cells (BHK, ATCC CCL 10); mouse sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TR1 cells (Mather et al., Annals N.Y. Acad. Sci. 383: 44-68 (1982)); MRC 5 cells or FS4 cells; or mammalian myeloma cells.

Host cells can be transformed or transfected with nucleic acids or vectors for polypeptide production and cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of polypeptides.

The host cells described herein may be cultured in a variety of media. Commercially available media such as Ham's F10 (Sigma), Minimal Essential Medium ((MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz., 58: 44 (1979), Barnes et al., Anal. Biochem., 102: 255 (1980), U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Upon culturing the host cells, the polypeptide can be produced intracellularly, in the periplasmic space, or directly secreted into the medium. If the polypeptide is produced intracellularly, as a first step, the particulate debris, either host cells or lysed fragments, is removed, for example, by centrifugation or ultrafiltration.

The compounds described herein largely may be made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

In various embodiments, a vector capable of expressing the peptides in an appropriate host is provided. The vector comprises the DNA molecule that codes for the peptides operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The polypeptide can be purified using, for example, hydroxylapatite chromatography, cation or anion exchange chromatography. Other techniques for protein purification such as ethanol precipitation, Reverse Phase HPLC, chromatofocusing, SDS-PAGE, and ammonium sulfate precipitation are also possible depending on the antibody to be recovered.

The term “Fc” refers to molecule or sequence comprising the sequence of a non-antigen-binding fragment resulting from digestion of whole antibody, whether in monomeric or multimeric form. The original immunoglobulin source of a native Fc is in one aspect of human origin and may be any of the immunoglobulins. A native Fc is a monomeric polypeptide that may be linked into dimeric or multimeric forms by covalent association (i.e., disulfide bonds), non-covalent association or a combination of both. The number of intermolecular disulfide bonds between monomeric subunits of native Fc molecules ranges from one to four depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG. Ellison et al., (1982), Nucleic Acids Res., 10: 4071-9. The term “native Fc” or “Fc” as used herein is generic to the monomeric, dimeric, and multimeric forms.

The term “Fc variant” refers to a molecule or sequence that is modified from a native Fc, but preferably still comprises a binding site for the salvage receptor, FcRn. International applications WO 97/34631 (published 25 Sep. 1997) and WO 96/32478 describe exemplary Fc variants, as well as interaction with the salvage receptor, and are hereby incorporated by reference. In one aspect, the term “Fc variant” comprises a molecule or sequence that is humanized from a non-human native Fc. In another aspect, a native Fc comprises sites that may be removed because they provide structural features or biological activity that are not required for the fusion molecules of interest. Thus, the term “Fc variant” comprises a molecule or sequence that lacks one or more native Fc sites or residues that affect or are involved in (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, (7) binding to the FcRn salvage receptor in cases where a shorter half-life is desired, or (8) antibody-dependent cellular cytotoxicity (ADCC).

Both native Fc's and Fc variants can be suitable Fc domains and can be used in various embodiments. A native Fc may be extensively modified to form an Fc variant provided binding to the salvage receptor is maintained; see, for example WO 97/34631 and WO 96/32478. In such Fc variants, one may remove one or more sites of a native Fc that provide structural features or functional activity not required by the fusion molecules of this invention. One may remove these sites by, for example, substituting or deleting residues, inserting residues into the site, or truncating portions containing the site. The inserted or substituted residues may also be altered amino acids, such as peptidomimetics or D-amino acids.

In various embodiments described herein, monoclonal antibodies can be obtained from a population of substantially homogeneous antibodies, i.e., the individual antibodies comprising the population are identical except for possible naturally occurring mutations or alternative post-translational modifications that may be present in minor amounts, whether produced from hybridomas or recombinant DNA techniques. Nonlimiting examples of monoclonal antibodies include murine, rabbit, rat, chicken, chimeric, humanized, or human antibodies, fully assembled antibodies, multispecific antibodies (including bispecific antibodies), antibody fragments that can bind an antigen (including, Fab′, F′(ab)₂, Fv, single chain antibodies, diabodies), maxibodies, nanobodies, and recombinant peptides comprising the foregoing as long as they exhibit the desired biological activity, or variants or derivatives thereof. Humanizing or modifying antibody sequence to be more human-like is described in, e.g., Jones et al., Nature, 321:522 525 (1986); Morrison et al., Proc. Natl. Acad. Sci., U.S.A., 81:6851 6855 (1984); Morrison and Oi, Adv. Immunol., 44:65 92 (1988); Verhoeyer et al., Science, 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al., (1994), J. Immunol., 152, 2968-2976; Studnicka et al., Protein Engineering, 7: 805-814 (1994); each of which is incorporated herein by reference in its entirety. One method for isolating human monoclonal antibodies is the use of phage display technology. Phage display is described in e.g., Dower et al., WO 91/17271, McCafferty et al., WO 92/01047, and Caton and Koprowski, Proc. Natl. Acad. Sci., USA, 87:6450-6454 (1990), each of which is incorporated herein by reference in its entirety. Another method for isolating human monoclonal antibodies uses transgenic animals that have no endogenous immunoglobulin production and are engineered to contain human immunoglobulin loci. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); WO 91/10741, WO 96/34096, WO 98/24893, or U.S. patent application publication nos. 20030194404, 20030031667 or 20020199213; each incorporated herein by reference in its entirety.

An “isolated” antibody refers to an antibody, as that term is defined herein, that has been identified and separated from a component of its natural environment. Contaminant components of its natural environment are materials that would interfere with diagnostic or therapeutic uses for the antibody, and may include enzymes, hormones, and other proteinaceous or nonproteinaceous solutes. In certain embodiments, the antibody will be purified (1) to greater than 95% by weight of antibody, and most preferably more than 99% by weight, (2) to a degree sufficient to obtain at least 15 residues of N-terminal or internal amino acid sequence, or (3) to homogeneity by SDS-PAGE under reducing or nonreducing conditions using Coomassie blue or, preferably, silver stain. Isolated naturally occurring antibody includes the antibody in situ within recombinant cells since at least one component of the antibody's natural environment will not be present. Ordinarily, however, isolated antibody will be prepared by at least one purification step.

An “immunoglobulin” or “native antibody” is a tetrameric glycoprotein. In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a “variable” (“V”) region of about 100 to 110 or more amino acids primarily responsible for antigen recognition. The carboxy-terminal portion of each chain defines a constant region primarily responsible for effector function. Immunoglobulins can be assigned to different classes depending on the amino acid sequence of the constant domain of their heavy chains. Heavy chains are classified as mu (μ), delta (Δ), gamma (γ), alpha (α), and epsilon (ε), and define the antibody's isotype as IgM, IgD, IgG, IgA, and IgE, respectively. Several of these may be further divided into subclasses or isotypes, e.g. IgG1, IgG2, IgG3, IgG4, IgA1 and IgA2. Different isotypes have different effector functions; for example, IgG1 and IgG3 isotypes have antibody-dependent cellular cytotoxicity (ADCC) activity. Human light chains are classified as kappa (10 and lambda (2) light chains. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989)).

For a detailed description of the structure and generation of antibodies, see Roth, D. B., and Craig, N. L., Cell, 94:411-414 (1998), herein incorporated by reference in its entirety. Briefly, the process for generating DNA encoding the heavy and light chain immunoglobulin sequences occurs primarily in developing B-cells. Prior to the rearranging and joining of various immunoglobulin gene segments, the V, D, J and constant (C) gene segments are found generally in relatively close proximity on a single chromosome. During B-cell-differentiation, one of each of the appropriate family members of the V, D, J (or only V and J in the case of light chain genes) gene segments are recombined to form functionally rearranged variable regions of the heavy and light immunoglobulin genes. This gene segment rearrangement process appears to be sequential. First, heavy chain D-to-J joints are made, followed by heavy chain V-to-DJ joints and light chain V-to-J joints. In addition to the rearrangement of V, D and J segments, further diversity is generated in the primary repertoire of immunoglobulin heavy and light chains by way of variable recombination at the locations where the V and J segments in the light chain are joined and where the D and J segments of the heavy chain are joined. Such variation in the light chain typically occurs within the last codon of the V gene segment and the first codon of the J segment. Similar imprecision in joining occurs on the heavy chain chromosome between the D and J_{H} segments and may extend over as many as 10 nucleotides. Furthermore, several nucleotides may be inserted between the D and J_{H} and between the V_{H} and D gene segments which are not encoded by genomic DNA. The addition of these nucleotides is known as N-region diversity. The net effect of such rearrangements in the variable region gene segments and the variable recombination which may occur during such joining is the production of a primary antibody repertoire.

The term “effective amount” refers to a dosage or amount that is sufficient to regulate iron transport, hemoglobin concentration or other characteristic of interest to achieve a desired biological outcome.

The term “therapeutic agent” is a substance that treats or assists in treating a medical disorder. As used herein, a therapeutic agent refers to a substance, e.g. IL-22-Fc, when administered to a subject along with, optionally, a composition described herein provides a better treatment compared to administration of the therapeutic agent or that composition alone. Non-limiting examples and uses of therapeutic agents are described herein.

The term “pharmacologically active” means that a substance so described is determined to have activity that affects a medical parameter (e.g., blood pressure, red blood cell count, cholesterol level, hematocrit, hemoglobin concentration) or disease state (e.g., iron disorders, cancer, autoimmune disorders). Thus, pharmacologically active peptides can comprise agonistic or mimetic or antagonistic peptides.

As used herein, a “therapeutically effective amount” refers to an amount which is effective, upon single or multiple dose administration to a subject (such as a human patient or other mammal) at treating, preventing, curing, delaying, reducing the severity of, ameliorating at least one symptom of a disorder or recurring disorder, or, prolonging the survival of the subject beyond that expected in the absence of such treatment.

Therapeutically effective amounts of a IL-22-Fc composition will vary and depend on the disease and the severity of the disease being treated and the weight and general state of the subject being treated, but generally range from about 1.0 ng/kg to about 100 mg/kg body weight, or about 10 ng/kg to about 30 mg/kg, or about 0.1 mg/kg to about 10 mg/kg or about 1 mg/kg to about 10 mg/kg per application. Administration can be daily, on alternating days, weekly, twice a month, monthly or more or less frequently, as necessary depending on the response to the disorder or condition and the subject's tolerance of the therapy. Maintenance dosages over a longer period of time, such as 4, 5, 6, 7, 8, 10 or 12 weeks or longer may be needed until a desired suppression of disorder symptoms occurs, and dosages may be adjusted as necessary. The progress of this therapy is easily monitored by conventional techniques and assays.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

The hepcidin-activity-affecting-composition can be administered by any suitable means, either systemically or locally, including via parenteral, subcutaneous, intraperitoneal, intrapulmonary, and intranasal, and, if desired for local treatment, intralesional administration. Parenteral routes include intravenous, intraarterial, intraperitoneal, epidural, intrathecal administration. Preferably dosing is given by injections, most preferably intravenous or subcutaneous injections, depending in part on whether the administration is brief or chronic. Other administration methods are contemplated, including topical, particularly transdermal, transmucosal, rectal, oral, local administration e.g. through a catheter placed close to the desired site or continuously via an infusion pump.

The term “treatment” refers to a therapeutic or preventative measure. The treatment may be administered to a subject having a medical disorder or who ultimately may acquire the disorder, in order to prevent, cure, delay, reduce the severity of, or ameliorate one or more symptoms of a disorder or recurring disorder, or in order to prolong the survival of a subject beyond that expected in the absence of such treatment.

The term “physiologically acceptable salts” comprises any salts that are known or later discovered to be pharmaceutically acceptable. Some possible examples are: acetate; trifluoroacetate; hydrohalides, such as hydrochloride and hydrobromide; sulfate; citrate; tartrate; glycolate; and oxalate.

In various embodiments, methods of using pharmaceutical compositions of IL-22-Fc are provided. Such pharmaceutical compositions may be for administration for injection, or for oral, pulmonary, nasal, transdermal or other forms of administration. In general, the invention encompasses pharmaceutical compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712. The compositions may be prepared in liquid form, or may be in dried powder, such as lyophilized form. Implantable sustained release formulations are also contemplated, as are transdermal formulations.

In various embodiments, the compounds may be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc., 85: 2149; Davis et al. (1985), Biochem. Intl., 10: 394-414; Stewart and Young (1969), Solid Phase Peptide Synthesis; U.S. Pat. No. 3,941,763; Finn et al. (1976), The Proteins (3rd ed.) 2:105-253; and Erickson et al. (1976), The Proteins (3rd ed.) 2:257-527. Solid phase synthesis is a preferred technique of making individual peptides since it is the most cost-effective method of making small peptides.

The compounds in one aspect are peptides, and they may be prepared by standard synthetic methods or any other methods of preparing peptides. The compounds that encompass non-peptide portions may be synthesized by standard organic chemistry reactions, in addition to standard peptide chemistry reactions when applicable.

Phage display, in particular, is useful in generating peptides for use in the present invention. It has been stated that affinity selection from libraries of random peptides can be used to identify peptide ligands for any site of any gene product. Dedman et al., J. Biol. Chem., 268: 23025-30, 1993. Phage display is particularly well suited for identifying peptides that bind to such proteins of interest as cell surface receptors or any proteins having linear epitopes. Wilson et al., Can. J. Microbiol., 44: 313-29, 1998; Kay et al., (1998), Drug Disc. Today, 3: 370-8. Such proteins are extensively reviewed in Herz et al. (1997), J. Receptor & Signal Transduction Res., 17(5): 671-776. Such proteins of interest are contemplated for use in this invention.

Peptide compounds are contemplated wherein all of the amino acids have a D configuration, or at least one of the amino acids has a D configuration. It is also contemplated that the peptide compounds may be cyclic.

Compounds that contain derivatized peptides or which contain non-peptide groups may be synthesized by well-known organic chemistry techniques.

All other technical terms used herein have the same meaning as is commonly used by those skilled in the art to which the present invention belongs.

Hepcidin is the central regulator of iron trafficking in humans. Extensive data from animal models and patients demonstrates that hepcidin controls dietary iron uptake and mobilization of iron from tissue stores (Sasu, B. J. et al. “Anti-hepcidin antibody treatment modulates iron metabolism and is effective in a mouse model of inflammation-induced anemia,” (Blood, In press 2009); Roetto, A. et al., Nat. Genet., 33, 21-22, 2003).

Elevated hepcidin causes reduced iron uptake from the diet and decreased iron release from the tissues resulting in decreased serum iron availability. As iron is a required growth factor for cellular and microbial proliferation, hepcidin-mediated iron withholding may reduce microbe (Weinberg, E. D. J. Infect. Dis., 124, 401-410, 1971).and tumor proliferation (Kalinowski, D. S. & Richardson, D. R, Pharmacol. Rev., 57, 547-583, 2005) in vivo.

An additional role for hepcidin-mediated iron modulation may be in the treatment of hereditary hemochromatosis (HH), hemolytic anemia and some forms of thalassemia. HH is the most common inherited disease in Caucasians and is characterized by abnormally low hepcidin expression (Papanikolaou, G. et al., Blood, 105, 4103-4105, 2005). The lack of hepcidin translates to excessive iron uptake from the diet and release from tissues leading to iron overload. Studies done using animal models of HH suggest that disease could be moderated by increased hepcidin availability (Viatte, L et al., Blood, 107:2952-2958, 2006). Individuals with hemolytic anemia and some forms of thalassemia also have abnormally low levels of hepcidin (Papanikolaou, G. et al., Blood 105, 4103-4105, 2005) and continue to absorb excessive amounts of dietary iron despite already having greatly overloaded iron stores. These patients may benefit from hepcidin-induced reduction in dietary iron uptake.

Because hepcidin may have a role in several disease states, treatment with this molecule for those in need of such a treatment may be desirable. Treatment, however, with recombinant hepcidin may be difficult. Therefore, an alternative treatment is to increase hepcidin expression or activity by stimulating an upstream regulator. Various embodiments in this patent are drawn, inter alia, to such a treatment.

Although the applicants invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicants reserve the right to exercise amendment rights under applicable patent laws to redefine the subject matter of such a claim to specifically exclude such statutory prior art or obvious variations of statutory prior art from the scope of such a claim. Embodiments defined by such amended claims also are contemplated.

### EXAMPLES

### Example 1

In order to investigate, the effects of IL-22 on hepcidin and iron transport IL-22-Fc was produced from HEK293-6E host cells and purified over a Protein A column. The Fc was attached to the N-terminal end of the IL-22 molecule. The amino acid and nucleic acid sequence information is provided below. In SEQ ID NOS. 1 and 2, Italics represent the VH5 leader sequence. Underlining represents the mouse Fc sequence. Bold represents the linker sequence and regular type represents the mouse IL-22 sequence. SEQ. ID. NO. 3 provides the nucleic acid sequence.

Full Amino Acid Sequence
[SEQ ID NO: 1]
MGSTAILGLLLAVLQGGRACKPCICTVPEVSSVFIFPPKPKDVLTITLTP
KVTCVVVDISKDDPEVQFSWFVDDVEVHTAQTQPREEQFNSTFRSVSELP
IMHQDWLNGKEFKCRVNSAAFPAPIEKTISKTKGRPKAPQVYTIPPPKEQ
MAKDKVSLTCMITDFFPEDITVEWQWNGQPAENYKNTQPIMDTDGSYFVY
SKLNVQKSNWEAGNTFTCSVLHEGLHNHHTEKSLSHSPGKGGGGSQEANA
LPVNTRCKLEVSNFQQPYIVNRTFMLAKEASLADNNTDVRLIGEKLFRGV
SAKDQCYLMKQVLNFTLEDVLLPQSDRFQPYMQEVVPFLTKLSNQLSSCH
ISGDDQNIQKNVRRLKETVKKLGESGEIKAIGELDLLFMSLRNACV
Predicted Mature Amino Acid Sequence
[SEQ ID NO: 2]
CKPCICTVPEVSSVFIFPPKPKDVLTITLTPKVTCVVVDISKDDPEVQFS
WFVDDVEVHTAQTQPREEQFNSTFRSVSELPIMHQDWLNGKEFKCRVNSA
AFPAPIEKTISKTKGRPKAPQVYTIPPPKEQMAKDKVSLTCMITDFFPED
ITVEWQWNGQPAENYKNTQPIMDTDGSYFVYSKLNVQKSNWEAGNTFTCS
VLHEGLHNHHTEKSLSHSPGKGGGGSQEANALPVNTRCKLEVSNFQQPYI
VNRTFMLAKEASLADNNTDVRLIGEKLFRGVSAKDQCYLMKQVLNFTLED
VLLPQSDRFQPYMQEVVPFLTKLSNQLSSCHISGDDQNIQKNVRRLKETV
KKLGESGEIKAIGELDLLFMSLRNACV
DNA Sequence
[SEQ ID NO: 3]
atggggtcaaccgccatccttggcctcctcctggctgtcctgcagggagg
gcgcgcctgtaagccttgcatatgtacagtcccagaagtatcatctgtct
tcatcttccccccaaagcccaaggatgtgctcaccattactctgactcct
aaggtcacgtgtgttgtggtagacatcagcaaggatgatcccgaggtcca
gttcagctggtttgtagatgatgtggaggtgcacacagctcagacgcaac
cccgggaggagcagttcaacagcactttccgctcagtcagtgaacttccc
atcatgcaccaggactggctcaatggcaaggagttcaaatgcagggtcaa
cagtgcagctttccctgcccccatcgagaaaaccatctccaaaaccaaag
gcagaccgaaggctccacaggtgtacaccattccacctcccaaggagcag
atggccaaggataaagtcagtctgacctgcatgataacagacttcttccc
tgaagacattactgtggagtggcagtggaatgggcagccagcggagaact
acaagaacactcagcccatcatggacacagatggctcttacttcgtctac
agcaagctcaatgtgcagaagagcaactgggaggcaggaaatactttcac
ctgctctgtgttacatgagggcctgcacaaccaccatactgagaagagcc
tctcccactctcctggtaaaggaggcggtggaagccaggaggcaaatgcg
ctgcccgtcaacacccggtgcaagcttgaggtgtccaacttccagcagcc
gtacatcgtcaaccgcacctttatgctggccaaggaggccagccttgcag
ataacaacacagacgtccggctcatcggggagaaactgttccgaggagtc
agtgctaaagatcagtgctacctgatgaagcaggtgctcaacttcaccct
ggaagacgttctgctcccccagtcagacaggttccagccctacatgcagg
aggtggtacctttcctgaccaaactcagcaatcagctcagctcctgtcac
atcagcggtgacgaccagaacatccagaagaatgtcagaaggctgaagga
gacagtgaaaaagcttggagagagtggagagatcaaggcgattggggaac
tggacctgctgtttatgtctctgagaaatgcttgcgtc

While mouse sequences are presented and have been used in the examples, it is expected that in various embodiments human sequences can replace the mouse sequences. For example, SEQ ID NO:4 (IL-22-linker-Fc) and SEQ. ID. NO. 5 (Fc-linker-IL-22) can be used. The human sequences presented use the Fc sequence from an IgG₁ molecule, however, Fc's from IgG₂, IgG₃, IgG₄, IgA IgD, IgE and IgM can also be used. Underlining represents the human Fc sequence. Bold represents the linker sequence and regular type (not underlined) represents the human IL-22 sequence.

(SEQ. ID. NO. 4)
Leader sequence-QGGAAAPISSHCRLDKSNFQQPYITNRTFMLAKE
ASLADNNTDVRLIGEKLFHGVSMSERCYLMKQVLNFTLEEVLFPQSDRFQ
PYMQEVVPFLARLSNRLSTCHIEGDDLHIQRNVQKLKDTVKKLGESGEIK
AIGELDLLFMSLRNACIGGGGSDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK.
(SEQ. ID. NO. 5)
Leader sequence-DKTHTCPPCPAPELLGGPSVFLFPPKPKDTLMIS
RTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSF
FLYSKLTVDKSRWQQGNVFSCSVMHEALHNHYTQKSLSLSPGKGGGGSQG
GAAAPISSHCRLDKSNFQQPYITNRTFMLAKEASLADNNTDVRLIGEKLF
HGVSMSERCYLMKQVLNFTLEEVLFPQSDRFQPYMQEVVPFLARLSNRLS
TCHIEGDDLHIQRNVQKLKDTVKKLGESGEIKAIGELDLLFMSLRNACI

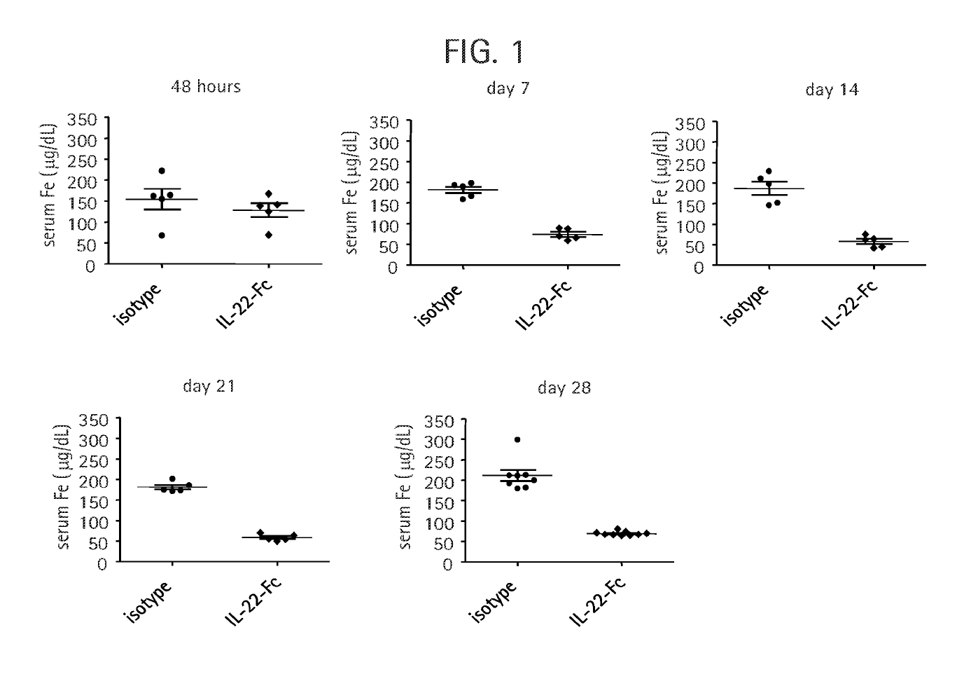

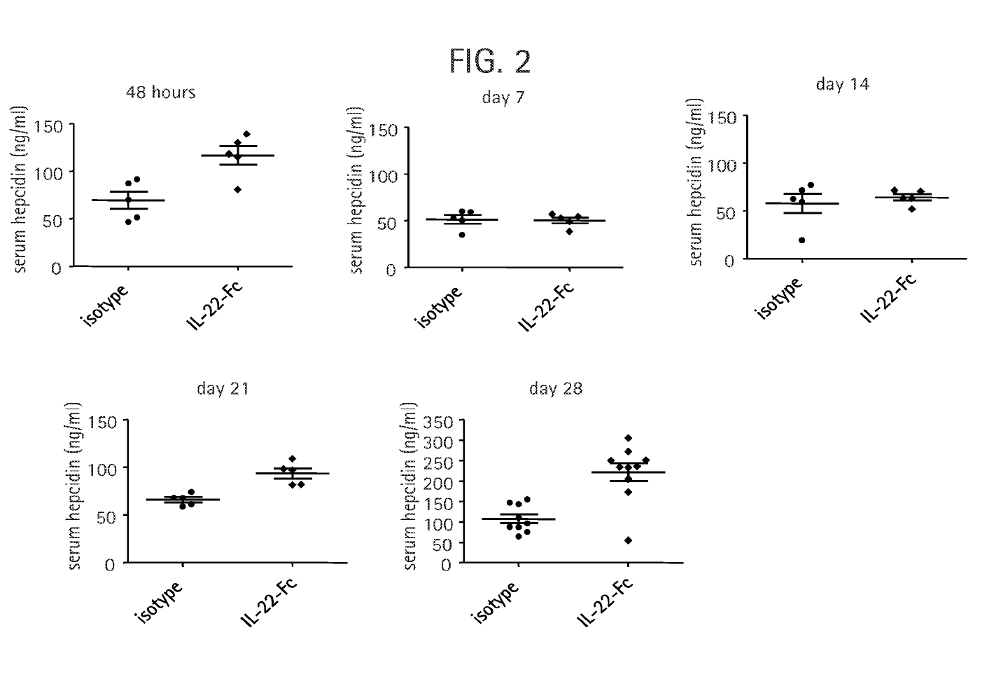

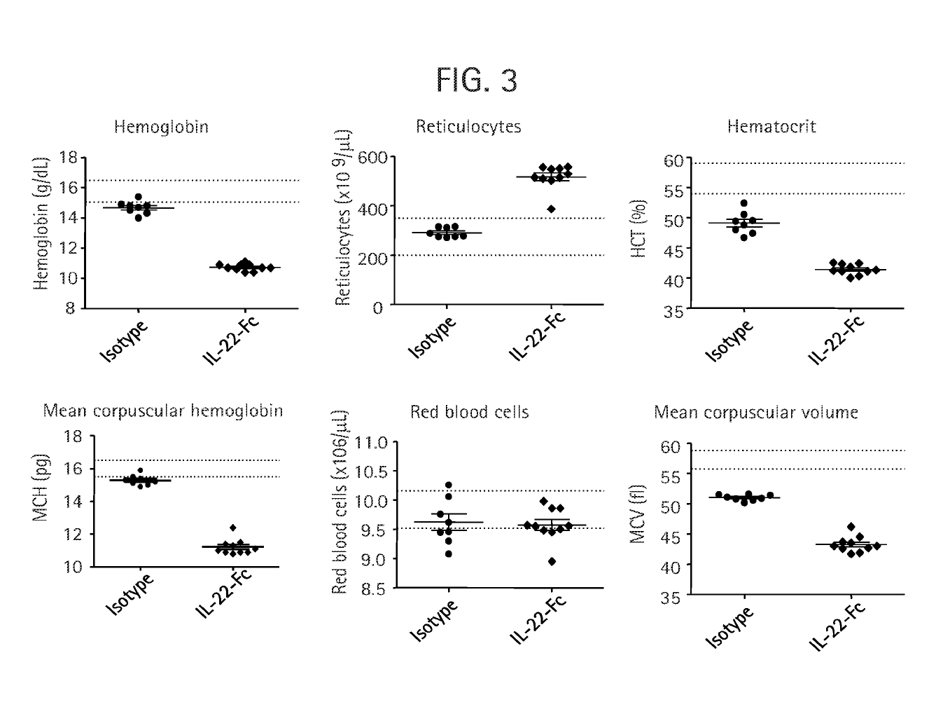

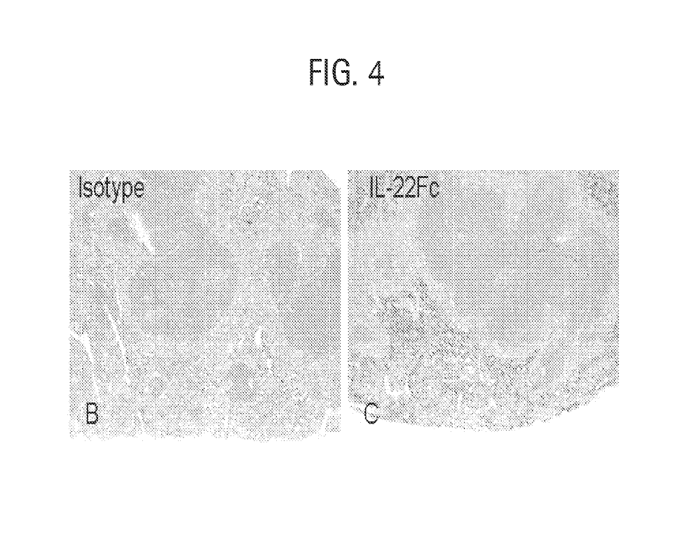

Female C57BL/6 mice were treated with an N-terminal Fc-conjugated form of IL-22 (IL-22-Fc) or mouse IgG1 isotype control protein (anti-AGP3 peptibody idiotype) for 28 days (150 μg IP 3×/week). Serum iron levels were determined using a standard clinical method (see Schade, A., et al., Proc. Soc. Exp. Biol Med. 87, 442, 1954 and Diehl, H. & Smith, G. F. “The Iron Reagents, Bathophenanthroline, 2,4,6-Tripyridyl-s-triazine, Phenyl-2-pyridylocine” GF Smith Chem. Co. 1960). Serum samples were measured using an Olympus AU400e clinical chemistry analyzer with Olympus Iron Reagent (Olympus Diagnostics, Melville, N.Y.). Data were measured at 48 hours and days 7, 14, 21 and 28. At 48 hours there was a statistically significant increase in serum hepcidin levels (116.5±10.0 ng/ml vs. 69.4±9.1 ng/ml for IL-22-Fc and isotype-treated mice, respectively) and a corresponding decrease in serum iron concentration (128.0±16.3 μg/dL vs. 154.2±24.7 μg/dL for IL-22-Fc and isotype-treated mice, respectively). Serum iron levels were consistently decreased throughout the study (FIG. 1 and Table 1) and serum hepcidin concentration was again significantly increased at day 28 (FIG. 2). As would be expected with hepcidin-restricted iron availability to the red blood cell compartment, the mice treated with IL-22-Fc had microcytic, hypochromic anemia (FIG. 3) and demonstrated increased iron accumulation in macrophage-rich regions of the spleen (FIG. 4).

TABLE 1

| | Serum iron (μg/dL) | | | | |
|---|---|---|---|---|---|
| | 48 hours | day 7 | day 14 | day 21 | day 28 |
| isotype | 154.2 ± 24.7 | 181.4 ± 7.7 | 187.2 ± 16.4 | 181.4 ± 5.5 | 211.3 +/− 13.4 |
| control | | | | | |
| IL-22-Fc | 128.0 ± 16.3 | 74.4 ± 6.0 | 57.6 ± 6.2 | 58.4 ± 3.8 | 69.3 +/− 14.7 |

Serum hepcidin levels were determined using a mass spectrometry-based method (Li et al., Journal of Pharmacological & Toxicological Methods, 2009, 59(3):171-180.) Data were measured at 48 hours and days 7, 14, 21 and 28. (FIG. 2 and Table 2—Mean±SEM.) The serum half life for hepcidin is short and may not be directly detectable at all time points. Hepcidin-mediated restriction in iron availability over the course of the experiment is apparent in the red blood cell parameters.

TABLE 2

| | Serum hepcidin (ng/ml) | | | | |
|---|---|---|---|---|---|
| | 48 hours | day 7 | day 14 | day 21 | day 28 |
| isotype | 69.4 +/− 9.1 | 51.5 ± 4.5 | 57.9 ± 10.2 | 66.1 ± 2.7 | 107.3 +/− 11.2 |
| control | | | | | |
| IL-22-Fc | 116.5 +/− 10.0 | 50.4 ± 3.2 | 64.1 ± 3.5 | 93.6 ± 5.3 | 221.4 +/− 21.6 |

Red blood cell and reticulocyte parameters indicate that IL-22-Fc-treated mice developed microcytic, hypochromic anemia (FIG. 3). Red blood cell and reticulocyte parameters were determined for C57BL/6 mice treated with IL-22-Fc or control protein for 28 days (150 μg IP 3×/week). Blood cell parameters were determined using a Bayer Advia 2120 hematology analyzer (Bayer Instruments, Tarry Town, N.Y.). Normal ranges for each parameter in female C57BL/6 mice are marked by dotted lines.

Iron accumulation is seen in macrophage-rich regions of the spleen (FIG. 4). Sections of the spleen were isolated from C57BL/6 mice treated with either IL-22-Fc or control protein for 28 days (150 μg IP 3×/week) and stained with Perl's iron stain. Results suggest that there are more macrophages in the IL-22-Fc-treated mice as compared to isotype control-treated mice and there are more iron deposits (blue) in the macrophage rich red pulp regions.

### Example 2

Female wild type B10 Q/Ai mice and mice naturally deficient in the signaling kinase Tyk2 (B10.D1-H2<q>tyke2<E775k>/J) were treated with an N-terminal Fc-conjugated form of IL-22 (IL-22-Fc) or isotype control protein (anti-AGP3 peptibody idiotype) for 28 days. The mice were injected IP with 150 μg 3-times per week.

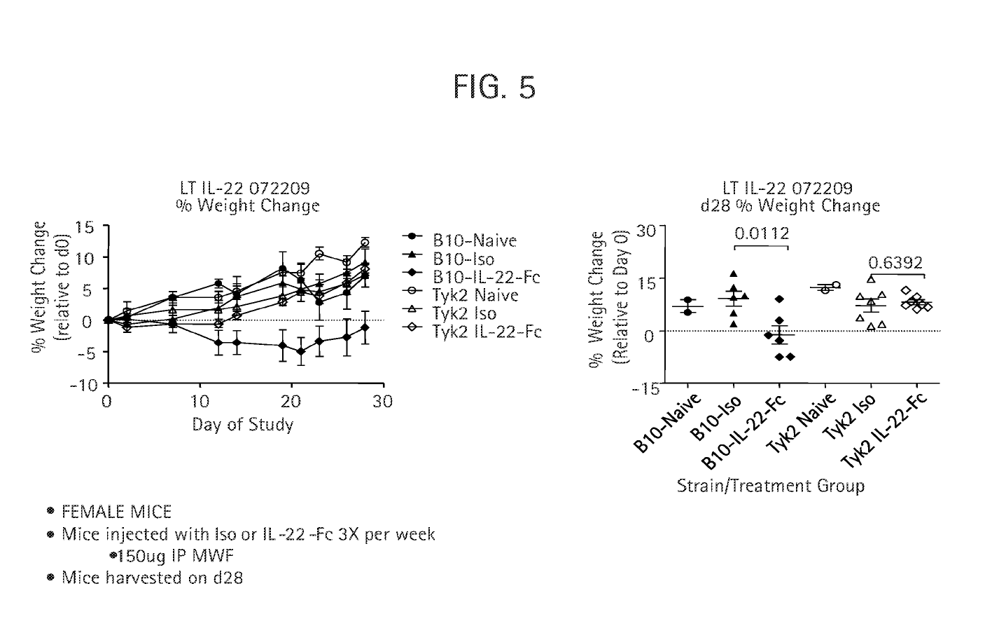

Over the course of 28 days, only the B10-IL-22-Fc mice lost weight following treatment (FIG. 5). This is also illustrated in FIG. 5 with a comparison of the relative weight change at day 28.

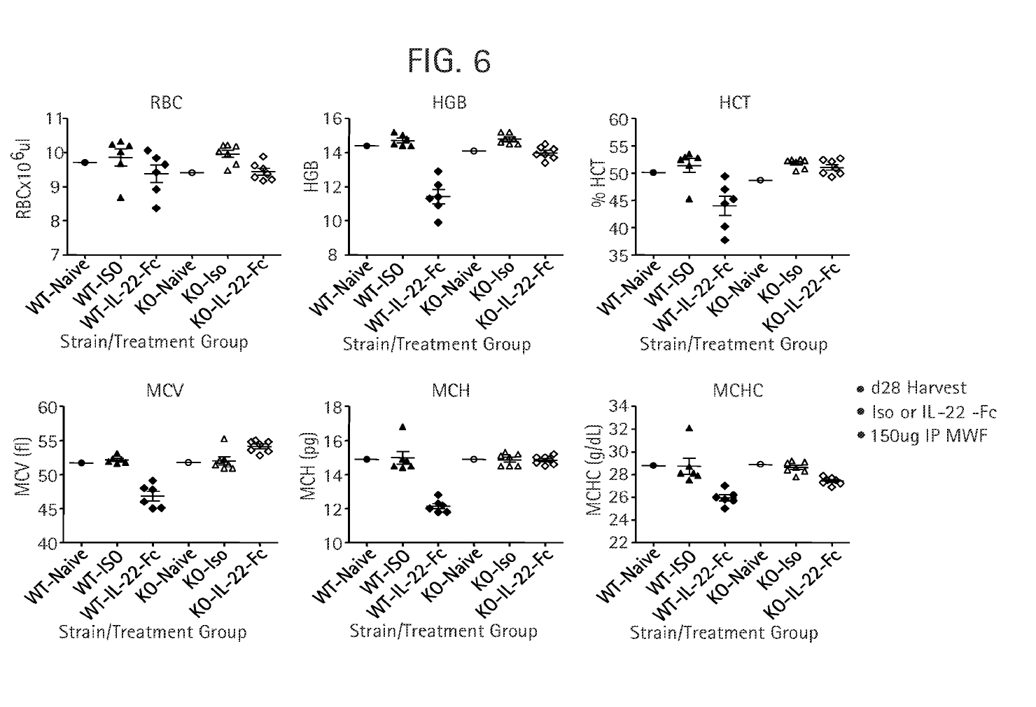

FIG. 6 illustrates differences in erythrocyte parameters at day 28 of treatment. It is clear that HGB, HCT, MCV, MCH and MCHC have all dropped following treatment with IL-22-Fc. This is also clearly illustrated in Table 3.

TABLE 3
All data analyzed using Prism software
Data reports mean +/− SEM
Stat analysis = unpaired t test

| | RBC | HGB | HCT | MCHC | MCH |
|---|---|---|---|---|---|
| WT Naive | 9.71 | 14.4 | 50.1 | 28.8 | 14.9 |
| WT ISO | 9.86 +/− 0.25 | 14.72 +/− 0.138 | 51.38 +/− 1.249 | 28.73 +/− 0.692 | 14.98 +/− 0.372 |
| WT IL-22-Fc | 9.38 +/− 0.26 | 11.42 +/− 0.418 | 43.98 +/− 1.769 | 25.95 +/− 0.268 | 12.15 +/− 0.154 |
| t test (Iso vs. IL-22-Fc) | 0.2118 | <0.0001 | 0.0066 | 0.0038 | <0.0001 |
| KO Naive | 9.41 | 14.1 | 48.7 | 28.9 | 14.9 |
| KO ISO | 9.96 +/− 0.11 | 14.8 +/− 0.113 | 51.79 +/− 0.326 | 28.63 +/− 0.191 | 14.87 +/− 0.136 |
| KO IL-22-Fc | 9.44 +/− 0.10 | 13.99 +/− 0.142 | 51.03 +/− 0.516 | 27.43 +/− 0.125 | 14.84 +/− 0.095 |
| t test (Iso vs. IL-22-Fc) | 0.0036 | 0.0008 | 0.2387 | 0.0002 | 0.8659 |
| ISO t test (WT vs. KO) | 0.6972 | 0.6462 | 0.7440 | 0.8783 | 0.7693 |
| IL-22-Fc t test (WT vs. KO) | 0.8192 | <0.0001 | 0.0018 | 0.0003 | <0.0001 |
| | MCV | MPV | RDW | # RETIC | % RETIC |
| WT Naive | 51.7 | 7 | 11.7 | 128.1 | 1.3 |
| WT ISO | 52.15 +/− 0.217 | 7.27 +/− 0.123 | 11.8 +/− 0.129 | 265.6 +/− 30.72 | 2.68 +/− 0.273 |
| WT IL-22-Fc | 46.83 +/− 0.695 | 7.72 +/− 0.192 | 20.12 +/− 0.692 | 851.2 +/− 141.1 | 9.283 +/− 1.74 |
| t test (Iso vs. IL-22-Fc) | <0.0001 | 0.0768 | <0.0001 | 0.0023 | 0.0038 |
| KO Naive | 51.8 | 6.8 | 12.4 | 260.1 | 2.8 |
| KO ISO | 52.03 +/− 0.563 | 7.27 +/− 0.106 | 11.76 +/− 0.087 | 262 +/− 16.55 | 2.643 +/− 0.156 |
| KO IL-22-Fc | 54.11 +/− 0.320 | 7.53 +/− 0.064 | 16.96 +/− 0.090 | 358.4 +/− 13.65 | 3.81 +/− 0.130 |
| t test (Iso vs. IL-22-Fc) | 0.0074 | 0.0608 | <0.0001 | 0.0007 | <0.0001 |
| ISO t test (WT vs. KO) | 0.8538 | 0.9770 | 0.7827 | 0.9166 | 0.8959 |
| IL-22-Fc t test (WT vs. KO) | <0.0001 | 0.3434 | 0.0005 | 0.0031 | 0.0069 |

RBC = Red Blood Cells (×10⁶/μl); HGB = hemoglobin (g/dl); HCT = hematocrit (%)
MCHC = Mean corpuscular hemoglobin concentration (g/dl); MCH = Mean corpuscular hemoglobin (per RBC - pg); MCV = mean corpuscular volume (fl); MPV = Mean platelet volume (fl); RDW = Red blood cell distribution width (%); #RETIC = number of reticulocytes; % RETIC = percent of reticulocytes.

### Example 3

Female C57BL/6 or C57BL/6 IL-6^{-/-} mice were treated with an N-terminal Fc-conjugated form of IL-22 (IL-22-Fc) or mouse IgG1 isotype control protein (anti-AGP3 peptibody idiotype) over a course of 28 days (150 μg IP 3×/week). Mice were harvested 4-5 hours post their final injection on days 2, 4, 7, 14, 21, or 28. Blood cell parameters were determined using a Bayer Advia 2120 hematology analyzer (Bayer Instruments, Tarry Town, N.Y.). Red blood cell and reticulocyte parameters were significantly reduced at 2 days post IL-22-Fc treatment in both C57BL/6 and C57BL/6^{-/-} mice and remained reduced at each time point evaluated. Percent iron content in the spleen was determined by scanning Perl's iron stained spleens using a Hamamtsu NanoZoomer Slide Scanner (Hamamatsu Corporation, Bridgewater, N.J.), producing a digital image of the entire microscope slide. Images of the spleens were analyzed with the Visiomorph Image Analysis Software system (Olympus America, Center Valley, Pa.) and the iron content as a percent of total splenic area was calculated. Iron content in the spleen was significantly increased at 2 days post IL-22-Fc treatment in both C57BL/6 and

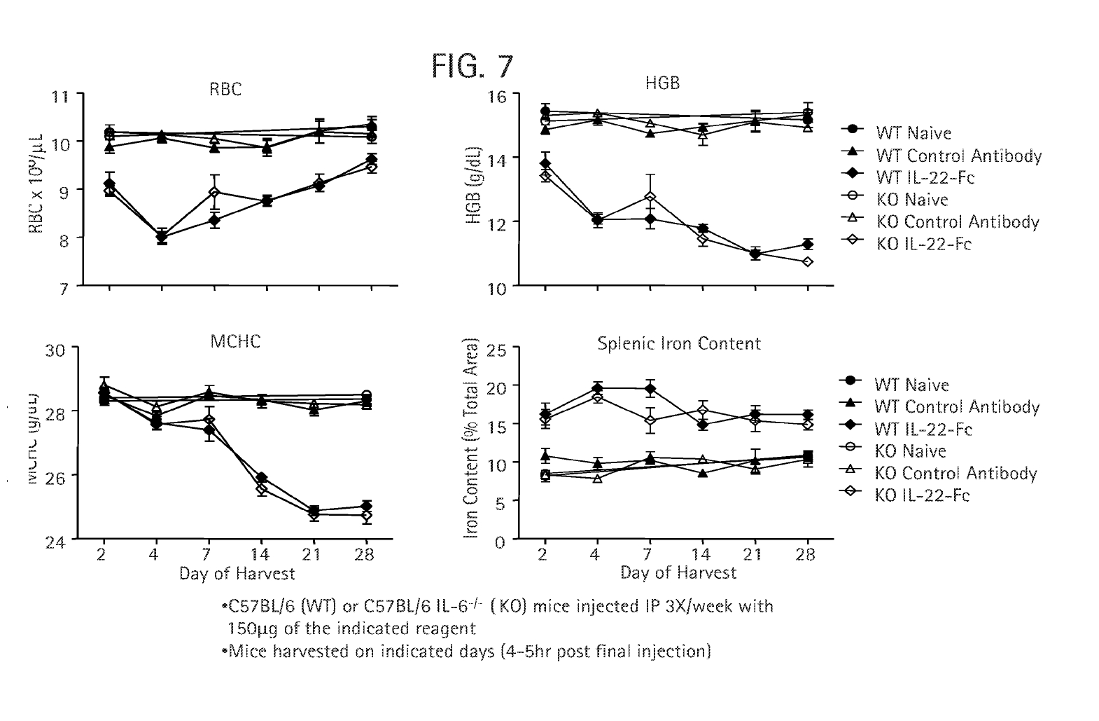

C57BL/6^{-/-} mice and remained elevated at each time point evaluated. These results suggest that IL-22 induced anemia and splenic iron accumulation is independent of IL-6. (FIG. 7)

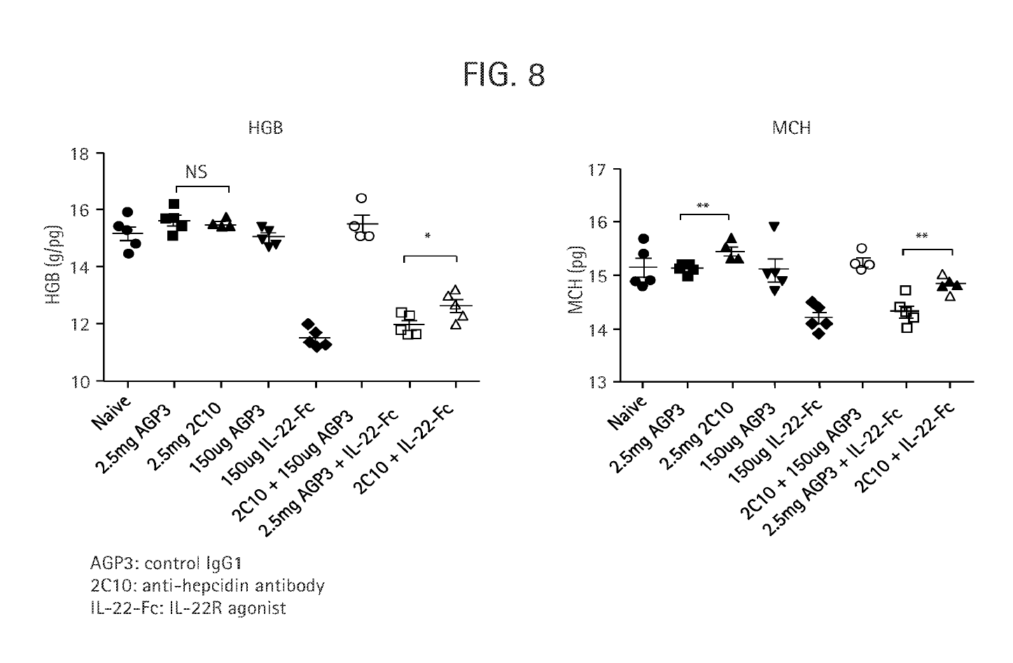

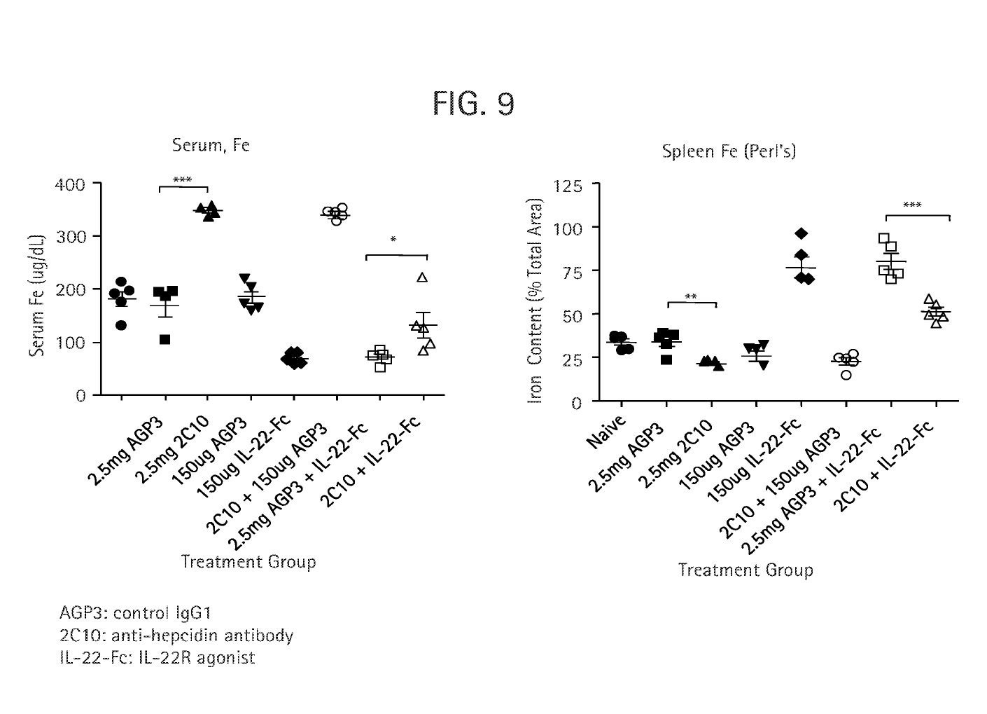

Indicated female C57BL/6 mice were treated with 2.5 mgs IP of a mouse IgG anti-hepcidin antibody (2C10) or mouse IgG isotype control protein (anti-AGP-3) on days −1, 1, 4, and 6. Indicated mice were treated with an N-terminal Fc-conjugated form of IL-22 (IL-22-Fc) or mouse IgG1 isotype control protein (anti-AGP3) with 150 ug IP on days 0, 2, 5, and 7. Mice were harvested 4-5 hours post the final injection of 150 ug of IL-22-Fc or control protein on day 7. Red blood cell and reticulocyte parameters were determined using a Bayer Advia 2120 hematology analyzer (Bayer Instruments, Tarry Town, N.Y.). Serum iron concentrations were measured using an Olympus AU400e clinical chemistry analyzer with Olympus Iron Reagent (Olympus Diagnostics, Melville, N.Y.). Percent iron content in the spleen was determined by scanning Perl's iron stained spleens using a Hamamtsu NanoZoomer Slide Scanner (Hamamatsu Corporation, Bridgewater, N.J.), producing a digital image of the entire microscope slide. Images of the spleens were analyzed with the Visiomorph Image Analysis Software system (Olympus America, Center Valley, Pa.) and the iron content as a percent of total splenic area was calculated. IL-22-Fc treatment reduced hemoglobin (HGB), mean corpuscular hemoglobin (MCH) and serum iron concentration while increasing the iron content of the spleen. Co-injection of anti-hepcidin antibody with IL-22-Fc resulted in an increase in HGB, MCH (FIG. 8), and serum iron (FIG. 9) with a corresponding decrease in splenic iron content. These results suggest blocking hepcidin can inhibit IL-22-Fc induced anemia and splenic iron accumulation.

Throughout this specification various publications, patents and patent applications have been referenced. The disclosures of these documents in their entireties are hereby incorporated by reference into this application. The reference to such documents, however, should not be construed as an acknowledgment that such documents are prior art to the application. Further, merely because a document may be incorporated by reference, this does not necessarily indicate that the applicants are in complete agreement with the document's contents.

Although various embodiments of the invention have been described with reference to various embodiments, those skilled in the art will readily appreciate that the specific examples and studies detailed above are only illustrative. It should be understood that various modifications can be made without departing from the spirit of the invention.

What is claimed:

1. A method of increasing hepcidin expression or activity in a mammal comprising stimulating the IL-22 pathway.

2. The method of claim 1, wherein the stimulating comprises treating with IL-22-Fc.

3. The method of claim 2, wherein the IL-22-Fc is comprised of amino acid sequence selected from the group consisting of SEQ ID NO:1, SEQ ID NO:2, SEQ ID NO:4 or SEQ ID NO:5.

4. The method of claim 3, wherein the Fc is fused to IL-22 at the N-terminal.

5. The method of claim 3, wherein the Fc is fused to IL-22 at the C-terminal.

6. The method of claim 1, wherein the increasing of hepcidin expression results in limiting iron uptake in a mammal.

7. The method of claim 6 wherein the mammal is a human.

8. The method of claim 6, wherein iron uptake is limited in hereditary or non-hereditary hemochromatosis, thalassemia, or hemolytic anemia.

9. An isolated amino acid sequence comprising SEQ ID NO:1, SEQ ID NO:2, SEQ ID NO:4 or SEQ ID NO:5.

10. An isolated amino acid sequence at least 95% identical to SEQ ID NO:1, SEQ ID NO:2, SEQ ID NO:4 or SEQ ID NO:5.

11. An isolated nucleic acid sequence encoding the isolated amino acid sequence of claim 9 or 10.

12. An isolated monoclonal antibody that binds the amino acid sequence of claim 9 or 10.

13. An agonist of an IL-22 receptor, wherein said agonist increases hepcidin expression or hepcidin activity.

14. The agonist of claim 13 comprising IL-22-Fc.

15. The agonist of claim 14, wherein the IL-22-Fc is comprised of amino acid sequence selected from the group consisting of SEQ ID NO:1, SEQ ID NO:2, SEQ ID NO:4 or SEQ ID NO:5.

16. The agonist of claim 14, wherein the Fc is fused to IL-22 at the N-terminal.

17. The agonist of claim 14, wherein the Fc is fused to IL-22 at the C-terminal
